(19) United States
(12) Patent Application Publication
(10) Pub. No.: US 2026/0345047 A1
(43) Pub. Date: Nov. 5, 2026

(54) System and Method for Detecting Acute Pain Using Spinal Cord EEG Signals

(71) Applicant: Ophelie Constant, Palo Alto, CA (US)

(72) Inventor: Ophelie Constant, Palo Alto, CA (US)

(21) Appl. No.: 19/198,041

(22) Filed: May 3, 2025

(51) Int. Cl. A61B 5/291; A61B 5/00; A61B 5/256; A61B 5/259; A61B 5/268; A61B 5/374

(52) U.S. Cl. CPC A61B 5/291; A61B 5/0006; A61B 5/256; A61B 5/259; A61B 5/268; A61B 5/374; A61B 5/4824; A61B 5/7207; A61B 5/7264; A61B 5/7455

(57) ABSTRACT

A non-invasive acquisition of 200-1200 Hz spinal HFOs over the lateral spinothalamic tract plus an HFO-tailored ML classifier. A wearable spinal-cord high frequencies oscillation (HFO) recorder system for detecting acute pain. The device comprises a flexible neckband with recording, reference and ground electrodes contacting skin over vertebrae C1-T1, electronics that digitise spinal-HFO signals, and a processor configured to extract high-frequency oscillations between 200 Hz and 1200 Hz. Firmware running on the processor and/or user equipment executes a machine-learning classifier trained to discriminate pain-related patterns from non-pain patterns and generates a real-time pain indication displayed numerically, visually, acoustically or haptically. The disclosure also covers a method of pain detection using the apparatus and a non-transitory computer-readable medium storing instructions that cause user equipment to analyse the digitised spinal-HFO data and output the pain indication.

### TECHNICAL FIELD

The present disclosure relates to bioelectronic monitoring devices and, more particularly, to a wearable spinal-cord high-frequency oscillation (HFO) system for detecting acute pain.

### BACKGROUND

Pain assessment in non-verbal patients and in remote or ambulatory settings remains subjective and unreliable. Electroencephalography of brain activity has been explored, but cervical spinal-cord signals contain rich nociceptive information that can be captured closer to the source. A practical, neck-worn sensor capable of acquiring and interpreting spinal-HFOs could provide objective, continuous pain monitoring.

### SUMMARY

In one aspect, a wearable spinal-cord HFO recorder apparatus is provided. The apparatus comprises a flexible neckband bearing a recording electrode assembly positioned over the lateral spinothalamic tract, on-board electronics that digitize the signals, and a processor configured to extract high-frequency oscillations (about 200-1 200 Hz±5%) indicative of acute pain. A wireless transceiver transmits the digitized data to user equipment executing a classifier that outputs a real-time pain indication.

In another aspect, a method of detecting acute pain using the apparatus is presented. The method records spinal-HFOs, identifies pain-related features, classifies the features and delivers a pain indication. A further aspect provides a non-transitory computer-readable medium containing instructions to implement the pain-state classifier.

A further aspect provides a non-transitory computer-readable medium containing instructions to implement the pain-state classifier.

### BRIEF DESCRIPTION OF THE DRAWINGS

FIG. 1 is a perspective view of the spinal-cord HFO neckband on a wearer.

FIG. 2 is a schematic of the electronic module and wireless transceiver.

FIG. 3 is a screenshot of a user-equipment interface displaying multi-channel spinal-HFO waveforms and a frequency spectrogram.

FIG. 4 is a drawing of the smartphone interface presenting a pain score and alert.

FIG. 5 is a drawing of the laptop interface displaying pain score and HFO signals.

FIG. 6 is a drawing of the placements on a subject's neck and connection to the electronics module.

FIG. 7 is an abstract functional diagram illustrating example components within the spinal cord EEG system in accordance with some embodiments;

### DETAILED DESCRIPTION

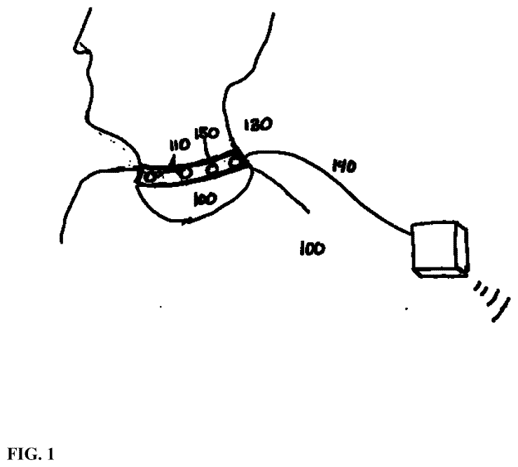

FIG. 1 is a schematic of the spinal cord EEG device 100 in accordance with some embodiments. The spinal cord EEG device is a bioelectronic interface comprised of one or more electrode sensors 110 that record and relay spinal cord neuron activity data to an electronic hardware component 120 suitable for biosignal acquisition. The electrode sensor(s) are configured within the spinal EEG device to contact the surface of the patient's skin in the cervical spine region 130, spanning between the first cervical vertebrae and the first thoracic vertebrae (C1-T1). The electrode sensor(s) may be configured in any way that enables detection and recording of spinal cord neuron activity. For example, the electrode sensor(s) may be placed at the back of the neck along the length of the spinal cord within the cervical spine region, or may be placed along the circumference of the neck within the cervical spine region. The electrode sensor(s) may be composed of any conductive material suitable for EEG recording, such as metals including silver, silver chloride, gold, stainless steel, or tin, encased in a flexible substrate such as silicone or soft plastic and may be of any size and dimension suitable for use within the cervical spine region. The electrode sensor(s) may be dry electrodes and utilize various conductive materials such as conductive polymers or metal microneedles on the skin contacting surface of the sensor(s) to enhance EEG signal conduction and reduce impedance. Alternatively, the electrode sensor(s) may utilize a conductive gel or paste applied between the conductive surface of the sensor(s) and the surface of the wearer's skin. The conductive gel or paste may be incorporated into the conductive surface of the electrode sensor(s) during manufacturing, or may be external to the spinal EEG device. Additionally, the electrode sensor(s) may incorporate a biocompatible adhesive to fix the the sensor(s) to the wearer's skin, especially in long-term monitoring situations. Reference and ground electrodes may be included in addition to recording electrodes. The electrode sensor(s) transmit recorded spinal EEG signals to the electronic hardware component through connection wires 140 with a pin on either side, one of which plugs into a port on the electrode sensor and the other into input channels within the electronic hardware component. In this embodiment the electrode sensor(s) may be fixed within a wearable device 150 that serves as a support structure and fastens the electrode sensor(s) in contact with the patient's skin within the cervical spine region. In this embodiment, the electrode sensor(s) may have the size and dimensions of standard electrodes used for brain EEG recording or may be of an alternative size and dimension. For instance the electrode sensor(s) may span the circumference of the wearable device creating a continuous conductive surface in contact with the wearer's skin, or may be spaced at regular intervals along the circumference of the wearable device, with non-conductive regions in between. In this embodiment, the electrode sensors may be dry electrodes, or may incorporate conductive gel into the skin-facing surface. The wearable device may be comprised of various materials or combinations of materials, both rigid and elastic, such as plastics, elastics, Velcro, rubber, metal, or textiles. These materials may be combined so that the wearable device is of fixed or adjustable circumference, and may use various types of fasteners to secure the device around the patient's neck, such as Velcro straps, buckles, snap fasteners, magnetic clasps, or elastic bands. The wearable device may also have varying dimensions, with a height, width, thickness, and weight within a range that facilitates fixation around the patient's neck and enables EEG signal recording. Additionally, the device may have a central passageway for the connection wires, consolidating the wires to a single path between the electrode(s) and the electronic hardware component. This embodiment is suitable for ease of use and long term spinal EEG recording.

Across the various embodiments of the spinal EEG device, the configuration of the electrode sensors may facilitate the recording of different sub-populations of spinal cord neurons. In the current embodiment, spinal cord neuron activity is recorded from the lateral spinothalamic tract, responsible for the detection of temperature and acute pain. In other embodiments, spinal cord neuron activity may be recorded from a single sub-population of spinal cord neurons, or from multiple sub-populations of spinal cord neurons. In the current embodiment, the electrode sensors record high frequency oscillations (HFOs) in the range of 200-1200 Hz. In other embodiments, the electrode sensors may record spinal cord activity data within standard frequency ranges between 1-100 Hz, or within higher frequency ranges between 200-1200 Hz.

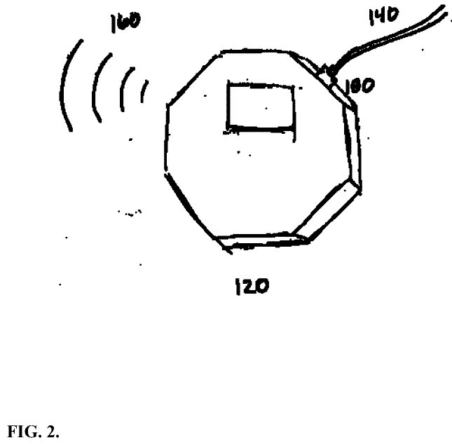

FIG. 2 depicts one embodiment of the electronic hardware component 120 of the spinal cord EEG device. In this embodiment, the electronic hardware component is a printed circuit board (PCB) with a plurality of channels that accept EEG signal inputs from the electrode sensor(s) through the connective wires 140. Alternatively, the electronic hardware could be any type electronic system that receives, encodes, and transmits EEG input data, such as a flexible electronic system, a 3D printed electronic system, or a system-on-a-chip (SoC). The electronic hardware component contains amplifiers to increase the magnitude of input EEG signals, filters to reduce or eliminate unwanted frequencies, a processor for initial processing such as signal normalization and encoding for transmission, and a power source. Additionally, the electronic hardware component contains an analog-to-digital converter (ADC) to convert the input analog EEG signals to digital signals. In the current embodiment, the electronic hardware component utilizes Bluetooth 160 to send the digitized EEG data wirelessly. Alternatively, the digitized EEG data can be sent wirelessly using Wi-Fi, or over wired connections such as universal serial bus (USB) or similar methods of wireless and wired data transmission.

In the current embodiment, the electronic hardware component includes a housing unit 170 that contains the PCB within an appropriately sized plastic housing with an internal impression to support and store batteries that power the PCB, as well as an opening 180 positioned in the top center of the plastic housing through which the connective wires pass facilitating communication between the PCB and the electrode sensor(s) of the spinal cord EEG device. In another embodiment, the electronic hardware component may be incorporated into the wearable spinal cord EEG device with the connective wires being contained within the support structure of the wearable device. In this embodiment, both the electronic hardware component and the connective wires are housed within the boundaries of the wearable spinal EEG device.

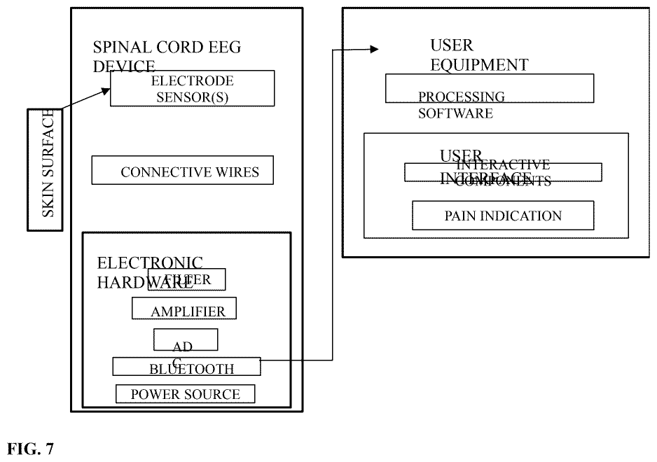

FIG. 7 is an abstract functional diagram illustrating example components within the spinal cord EEG system in accordance with some embodiments. As shown in the diagram, the system can be comprised of a spinal cord EEG device that is in contact with the wearer's skin in the cervical spine region and includes one or more electrode sensors that detect and record spinal cord EEG data, which is transmitted to an electronic hardware component via leads connected to a plurality of channels on the electronic hardware component. The electronic hardware component can include filters, amplifiers, an ADC, a battery or alternative power source, and data transmission capabilities, either wireless or wired. Spinal EEG data recorded from the electrode sensors within the spinal EEG device is transmitted from the electronic hardware component to a user equipment (UE) for further processing, analysis, and display, which is enabled through processing software supported by the UE. In the present embodiment, the user equipment is a laptop computer, capable of receiving the transmitted spinal EEG data and supporting processing software that processes the recorded spinal EEG signals and reports and indication of the wearer's pain status within a user interface (UI) as described in the present disclosure.

The processing software utilizes various signal processing tools to process the recorded spinal EEG signals and derive and a wearer pain indication that is signaled by the UI. The processing software may enable preprocessing of the EEG data such as filtering to remove noise and artifacts and segmentation to divide continuous EEG data into epochs or segment based on events or time intervals, in addition to other preprocessing techniques that prepare the spinal EEG data for further signal processing. Signal processing tools may be used to detect waveform patterns or a particular “pain pattern” indicative of the presence of acute pain. Differentiating characteristics of these waveform patterns may include specific spike patterns, spike-wave complex patterns, or waveform repetition, timing, and amplitude, in addition to other identifying waveform features. These waveform patterns may be specific to EEG signals within standard frequency ranges (1-100 Hz) or HFOs (200-1200 Hz). Additionally, the processing software may include data visualization tools to visualize the processed spinal EEG data, as well as a UI to enable user interaction with the spinal EEG data and provide an indiction of wearer pain status.

Processing techniques may include various methods of frequency analysis such as fourier transforms or wavelet transforms. Fourier transform may be used to decompose the spinal EEG signals into their frequency components and generate a frequency spectrum showing the amplitude of the different frequency components present in the EEG data. This may be used to categorize EEG frequencies into frequency bands, such as delta (1-4 Hz), theta (4-8 Hz), alpha (8-12 Hz), beta (12-30 Hz), gamma (30-100 Hz), and HFOs (200-1200 Hz), or other categories relevant to spinal EEG signal processing. Analysis of the derived frequency spectrum can be used to identify the pain state of the wearer based on the frequencies present. Alternatively, wavelet transform can be used to provide both frequency and time information of the spinal EEG signals. The time-frequency representation of the spinal EEG signals enables the analysis of dynamic changes in neural activity corresponding to state changes, such as changes in the wearer's pain status.

Alternatively, statistical analysis, alone or in combination with machine learning techniques may be used to analyze spinal cord EEG signal characteristics and determine differences between wearer pain states. Feature extraction can be used to extract any type of relevant spinal EEG signal features indicative of acute pain, such as various waveform patterns, power in specific frequency bands such as delta, theta, alpha, beta, gamma, or HFOs, time-domain characteristics, or any other signal features indicative of a “pain pattern,” indicating that the wearer is experiencing acute pain. Statistical analysis can be used to determine if there are significant differences between the extracted features and features of baseline spinal EEG data or to characterize the likelihood that a recorded spinal EEG pattern aligns with a predetermined “pain pattern.” In one embodiment, baseline spinal EEG data constitutes spinal EEG data collected from individuals with varying degrees of acute pain in order to generate a database of spinal EEG data for comparison and interpretation of the wearer's pain state. In another embodiment, in the case that the wearer will be undergoing a medical operation or procedure, baseline spinal EEG data is collected from the wearer preoperatively and is compared to spinal EEG data recorded from the wearer postoperatively using the spinal EEG device. Postoperative deviations from the baseline spinal EEG signals serve as an indication of the wearers pain state. Alternatively, the recorded spinal EEG data could be compared to a predetermined “pain pattern,” representing a known condition of acute pain.

Additionally, machine learning algorithms such as support vector machines, neural networks, or decision trees may be used to classify recorded spinal EEG data into different pain states based on extracted features, or classify the recorded spinal EEG signals as a “pain pattern” or “non-pain pattern,” or similar distinguishing terminology. In the case that a “pain pattern” is detected, a pain indication may be provided. The pain indication may be any signal indicating the pain state of the wearer. For instance, the pain indication can be a binary indication of the presence or absence of pain, or may be a “pain score” indicating the level of pain the wearer is experiencing. The “pain score” may be a score between 1 and 10 representing varying degrees of wearer pain, with 1 being minimal to no acute pain and 10 being intense acute pain. In the present embodiment, the pain indication is displayed visually on the screen of the UE within the UI environment. Alternatively, the pain indication may be an audio signal such as a beep or tone, a tactile alert such as a vibration, an alternative visual alert such as flashing LED lights or color-coded indicator lights, or a combination of the above alert mechanisms. The pain indication alerts the user to the wearer's pain status as a means of monitoring the wearer's pain status.

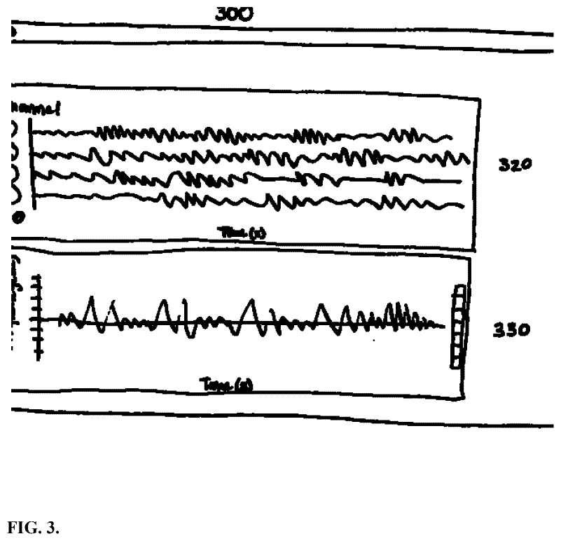

FIG. 3 is an illustration of the UI component 300 of the processing software displaying the processed spinal EEG data recorded from spinal EEG device and transmitted from the electronic hardware component. In the current embodiment, the UI is graphical user interface (GUI) comprised of a control panel that enables real time spinal EEG data streaming and processing. For each of the plurality of channels 310 supported by the electronic hardware, plots of the voltages of the recorded signals versus the elapsed time in seconds 320 is displayed, along with a plot of the detected frequencies (Hz) versus time (seconds) 330. Additional plots may include plots of amplitude, power, or other signal metrics. The UI may also incorporate interactive data visualization techniques, such as the option to apply filters to the data to visualize only certain frequencies of spinal EEG for example. Additionally the UI provides a pain indication to the user according to the embodiments described within this disclosure as a means of informing the user of the wearer's pain status. The UI may also include functionality to view changes in the wearer's pain status overtime, for instance, over the course of a prolonged pain monitoring period.

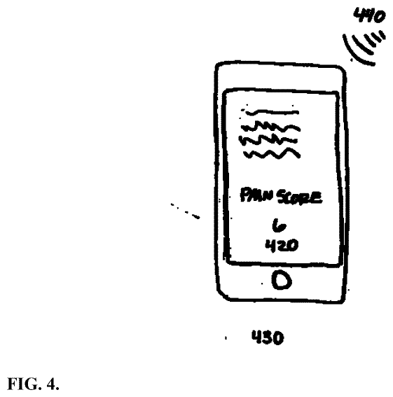

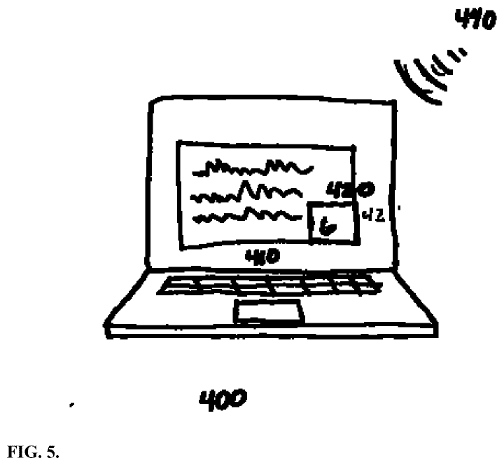

FIG. 4 and FIG. 5 are schematics of the UE displaying the recorded spinal EEG data wearer pain indication within a UI enabled by the processing software, using spinal EEG data transmitted wirelessly 440 from the spinal EEG device. In the current embodiment, as shown in FIG. 4A, the UE is a laptop computer 400 that supports the processing software component and the interactive UI as described in the present disclosure. Within the UI 410, a pain indication 420 is signaled visually in the screen as a score between 1 and 10 to inform the user of the wearer's pain status. In another embodiment, the UE may be a type of mobile computing device such as a smartphone, tablet, or wearable that may facilitate remote monitoring of a patient's pain status. FIG. 4B illustrates one embodiment of the UE as a smartphone 430 with a GUI displaying an indication of the patient's pain status. This may be used as a means of notifying a user to episodes of wearer pain to inform intervention.

FIG. 5 depicts a laptop interface displaying a pain score alongside HFO signal waveforms. This desktop interface provides a comprehensive view for detailed analysis, combining quantitative pain scoring with the raw HFO signal data for clinical evaluation.

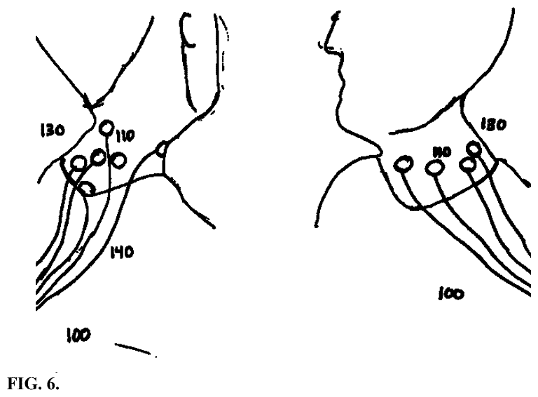

FIG. 6 demonstrates the electrode placement configuration on a subject's neck and the connection pathway to the electronics module. This view illustrates the practical application of the device, showing how the electrodes are positioned to capture spinal cord signals and how they interface with the processing electronics

Additionally, it depicts one embodiment of the spinal EEG device's electrode sensor(s) as described in the present disclosure. In this embodiment, the electrode sensor(s) are of standard size and dimension, similar to those used for standard brain EEG recording. The electrode sensor(s) are free and independently placed in various locations within the patient's cervical spine region, with connective wires that span a suitable length between the connection points at the electrode sensor(s) and the electronic hardware component and rest freely against the user. The electrode sensor(s) are fixed to the surface of the wearer's skin using a biocompatible adhesive material. In this embodiment, the electrode sensor's may be placed in any configuration or montage that enables recording and transmission of spinal cord EEG signals.

What is claimed is:

1. A non-invasive neck-worn spinal-cord high-frequency oscillation (HFO) monitoring system comprising: a flexible band configured to encircle the wearer's neck and to be positioned over vertebral levels C1-C7; a single annular recording electrode assembly carried by an inner surface of the band, the electrode assembly including a plurality of discrete contact points arranged in a ring configuration to contact the wearer's skin and detect HFO signals indicative of acute pain; at least one reference electrode and at least one ground electrode disposed on the band; an electronics module integrated with or tethered to the band, the electronics module including a plurality of input channels configured to receive HFO signals from the recording electrode assembly, amplifiers configured to amplify the received HFO signals, filters configured to remove unwanted frequencies and noise, an analog-to-digital converter configured to digitize the filtered HFO signals, a processor configured to extract high-frequency oscillations in a range of about 200 Hz to about 1 200 Hz (±5%) and to analyze the extracted HFOs using a machine-learning classifier, a power supply, and a wireless transceiver configured to transmit processed spinal-HFO data to user equipment; wherein the machine-learning classifier is trained to generate a pain indication representing a current acute-pain state of the wearer.

2. The system of claim 1, wherein the recording electrode assembly comprises dry electrodes comprising conductive polymers or metal microneedles on a skin-contacting surface to reduce skin-electrode impedance below 10 kΩ.

3. The system of claim 1, wherein the contact points of the recording electrode assembly comprise a conductive material selected from the group consisting of silver, silver chloride (500 μm Ag/AgCl micro-needles), gold, stainless steel and tin, wherein the conductive material is encased in a flexible substrate selected from silicone or soft plastic.

4. The system of claim 1, wherein the recording electrode assembly comprises at least one of: (a) a continuous conductive strip extending around at least 75% of the circumference of the band; or (b) discrete electrodes spaced at regular intervals along the circumference of the band with non-conductive regions therebetween.

5. The system of claim 1, further comprising a conductive gel or paste interface between the recording electrode assembly and the wearer's skin, wherein the conductive gel or paste is either pre-applied to the electrodes during manufacturing or applied at the time of use.

6. The system of claim 1, wherein the band comprises: (a) an adjustable circumference mechanism selected from the group consisting of hook-and-loop fasteners, buckles, snap fasteners, magnetic clasps and elastic sections; and (b) a material composition selected from the group consisting of medical-grade plastics, elastics, rubber, textiles and combinations thereof.

7. The system of claim 1, further comprising a housing unit containing the electronics module, the housing unit including a battery compartment and a central passageway for consolidating connection wires between the electrodes and the electronics module, wherein the housing unit is either separate from or integrated into the band structure.

8. The system of claim 1, wherein the electronics module is implemented as a system-on-chip (SoC) comprising the amplifiers, filters, analog-to-digital converter, processor and wireless transceiver integrated on a single substrate.

9. The system of claim 1, wherein the processor executes a machine-learning classifier configured to discriminate between pain patterns and non-pain patterns in the HFO frequency range, classify pain intensity on a numerical scale from 1 to 10 (where 1 represents minimal acute pain and 10 represents intense acute pain), and update classification parameters based on wearer-specific baseline data.

10. The system of claim 9, wherein the machine-learning classifier comprises at least one of a neural network, a support vector machine or a decision tree algorithm, trained using pre-operative baseline spinal-HFO data from the wearer and a database of spinal-HFO patterns from multiple subjects with varying degrees of acute pain.

11. The system of claim 1, wherein the processor is further configured to perform frequency-band analysis to categorize the spinal-HFO signals from about 200 Hz to 1 200 Hz (±5%) and identify pain-indicative patterns based on frequency-band power distributions.

12. The system of claim 1, wherein the pain indication comprises at least one of: a numerical pain score on a scale of 1 to 10 displayed on the user equipment; a binary pain/no-pain indicator; a color-coded visual alert corresponding to pain intensity levels; an audio signal with frequency or pattern corresponding to pain intensity; a tactile alert with vibration patterns corresponding to pain levels; and a time-series display showing pain level changes over a monitoring period.

13. The system of claim 1, wherein the wireless transceiver supports at least one of Bluetooth® Low Energy 5.2 with a range of at least 10 m, Wi-Fi (IEEE 802.11) or proprietary wireless protocols optimized for continuous biomedical data transmission.

14. The system of claim 1, wherein the user equipment comprises at least one of a laptop computer, a smartphone, a tablet or a dedicated medical monitoring device and includes a graphical user interface configured to display real-time voltage-versus-time waveforms for each electrode channel, frequency-versus-time spectrograms showing the HFO activity, a continuously updated pain-score indicator and historical pain-trend data over the monitoring period.

15. The system of claim 1, further comprising a biocompatible adhesive material on the electrode surfaces for securing long-term skin contact during extended monitoring periods.

16. A method for detecting acute pain in a human subject using spinal-cord HFO monitoring, the method comprising positioning the system of claim 1 around the subject's neck such that the recording electrode assembly contacts skin overlying the lateral spinothalamic tract between vertebrae C1 and C7; establishing a baseline spinal-HFO pattern by recording signals prior to an anticipated pain-inducing event; continuously recording spinal-HFO signals during and after the pain-inducing event; extracting high-frequency oscillations in the range of about 200-1 200 Hz (±5%) from the recorded signals; comparing the extracted oscillations to the baseline pattern to identify deviations indicative of acute pain; applying a trained machine-learning classifier to determine a pain-intensity level; and generating and displaying a real-time pain indication on the user equipment.

17. The method of claim 16, further comprising preprocessing the spinal-HFO signals by applying band-pass filtering to isolate the HFO frequency range, removing power-line interference at 50 or 60 Hz, segmenting the signals into epochs of 0.5 s to 2 s and rejecting artifacts caused by movement or electromagnetic interference.

18. The method of claim 16, further comprising performing time-frequency analysis using wavelet transforms to capture dynamic changes in HFO patterns, identifying transient HFO bursts correlated with acute-pain episodes and tracking the temporal evolution of pain patterns over the monitoring period.

19. The method of claim 16, wherein establishing the baseline comprises recording pre-operative spinal-HFO data for at least 10 minutes, extracting statistical features including mean power, peak frequency and HFO-burst rate (300-500 Hz), and creating a personalized baseline profile for the subject.

20. The method of claim 16, further comprising continuously updating the machine-learning classifier with newly labeled pain episodes to improve subject-specific accuracy, adjusting classification thresholds based on the subject's pain-sensitivity profile and generating alerts when pain levels exceed predetermined thresholds.

21. The method of claim 16, wherein the pain-inducing event comprises at least one of surgical procedures, medical interventions, diagnostic procedures or controlled pain stimuli for calibration.

22. The method of claim 16, further comprising transmitting pain-indication data to healthcare-provider systems for remote monitoring, electronic health records for documentation and pain-management systems for automated-intervention triggering.

23. A non-transitory computer-readable medium storing instructions that, when executed by a processor, cause a system to perform real-time acute-pain detection comprising receiving digitized spinal-HFO data from the system of claim 1; applying signal-processing algorithms to extract HFOs in the range of about 200-1 200 Hz (±5%) by wavelet entropy extraction, performing Fourier transforms to generate frequency spectra, performing wavelet transforms to generate time-frequency representations and calculating power spectral density for defined frequency bands; executing a trained machine-learning algorithm to classify HFO patterns as indicative of pain or no-pain states, determine pain intensity on a scale of 1 to 10 and identify pain-pattern trends over time; generating a graphical user interface displaying multi-channel real-time frequency spectrograms highlighting HFO activity, a prominent pain-score indicator updated in real time and historical pain trends and statistics; and triggering alerts when pain scores exceed configurable thresholds.

24. The computer-readable medium of claim 23, wherein the instructions further cause the system to maintain a database of baseline and historical spinal-HFO patterns for the subject, perform comparative analysis between current and baseline patterns, generate pain-trajectory predictions based on pattern evolution, provide recommendations for pain-management interventions and export pain-monitoring reports for clinical documentation.

25. The computer-readable medium of claim 23, wherein the instructions implement adaptive filtering algorithms that automatically adjust to subject-specific signal characteristics, compensate for electrode-impedance variations over time, enhance HFO signal-to-noise ratio and maintain stable pain-detection accuracy during long-term monitoring.
